(19) United States
(12) Patent Application Publication
(10) Pub. No.: US 2025/0213235 A1
(43) Pub. Date: Jul. 3, 2025

(54) Aspiration Needle with Venting Feature

(71) Applicant: Alec Goldenberg, New York, NY (US)

(72) Inventor: Alec Goldenberg, New York, NY (US)

(21) Appl. No.: 18/402,386

(22) Filed: Jan. 2, 2024

(51) Int. Cl. A61B 10/02

(52) U.S. Cl. CPC A61B 10/025; A61B 10/0283; A61B 2010/0258

(57) ABSTRACT

An aspirating needle for collecting a specimen includes an elongated body that includes a first lumen and a second lumen. The first lumen is open at both ends for placement at a specimen site to collect and permit aspiration of the specimen from the specimen site. The second lumen terminates at a distal vent port formed along the elongated body such that that when the needle is placed at the specimen site. The distal vent port is positioned at the specimen site. The needle includes a handle that extends radially outward from the elongated body to permit a force to be applied to the needle for directing the needle to the specimen site. The handle includes a vent channel that is in fluid communication with the second lumen. The aspirating needle includes an air reservoir that is open to atmosphere and includes a mechanism for limiting an amount of air that can be delivered to the specimen site by traveling within the second lumen and being discharged through the distal vent port.

### TECHNICAL FIELD

The present invention relates to an instrument, typically known as a needle or cannula that is used to gather a sample from a site using aspiration, and more particularly, relates to an aspiration needle for gathering tissue from living persons or animals for pathological study and includes an improved structure for collecting a fluid sample of bone marrow.

### BACKGROUND

For various medical reasons, such as diagnostic tests or the like, it is often necessary for a physician to obtain a sample of a specific tissue from a patient. Often, a biopsy (sample) is required from a rigid structure, such as a bone or bone marrow. Bone marrow biopsies are typically recovered with significant portions of their internal bony structure intact which allows the pathologist to provide interpretations regarding bone marrow cellularity or possible infiltration with abnormal cells.

A bone marrow sampling procedure usually includes both the collection of a core biopsy using a bone marrow biopsy needle and a fluid sample of bone marrow using an aspiration needle. The two specimens provide complementary information that is relevant for the evaluation of a variety of malignant and nonmalignant hematologic processes. The bone marrow aspiration provides a liquid sample of suspended hematopoietic progenitor cells, stromal cells, and trabecular bone fragments that can be processed for flow cytometric analysis of the bone marrow content, for cytogenetic studies, as well as for the preparation of smears for detailed morphologic evaluation of the progenitor cell morphology. The core biopsy provides accurate information regarding the status of the supporting bone, the cellularity of the bone marrow sample, and the identification of extrinsic cells as seen when the bone marrow is infiltrated with lymphoma or carcinoma.

The process of obtaining both the core biopsy and aspiration sample can produce significant pain for the patient. Specimen capturing needles, including those of the present applicant that are set forth in issued and pending applications, have been designed in an attempt to limit the manipulation of the bone marrow biopsy needle, to increase the recovery of more substantial specimens and to decrease patient pain. However, conventional needles have not been specifically designed to minimize the pain associated with the aspiration process.

Aspiration type needles have a relatively simple design. The needle typically has a sharp tip for puncturing the cortical bone and usually a hub and handle to facilitate the operators guiding the tip safely into the appropriate position. A stylet is left in place until the needle has penetrated the cortex, after which it is removed and an aspirating syringe is placed at the hub. The syringe plunger is rapidly withdrawn to quickly produce a negative pressure which is transmitted through the needle into the bone marrow space to dislodge the material and facilitate its collection into the syringe through the needle. The procedure of quickly pulling back on the plunger and producing a negative pressure usually produces significant pain often described as radiating down the leg. Since the advent of specimen capturing needles, the pain has been described by some patients as being worse than the pain associated with the bone marrow biopsy procedure. An aspirate needle that minimizes patient pain would make the bone marrow procedure more tolerable and acceptable. Moreover, an aspirate needle that minimizes pain would be especially advantageous when multiple aspirates are required to recover a sufficient quantity of bone marrow material for processing.

The exact mechanism that results in the pain and its radiation down into the lower extremity is unknown. It is hypothesized that the introduction of a negative pressure into the bone marrow space stimulates a variety of nerve fibers that results in the pain. Alternatively, simple disruption of the trabecular structure may be the source of the pain.

### SUMMARY

An aspirating needle for collecting a specimen includes an elongated body that includes a first lumen and a second lumen. The first lumen is open at both ends for placement at a specimen site to collect and permit aspiration of the specimen from the specimen site. The second lumen terminates at a distal vent port formed along the elongated body such that that when the needle is placed at the specimen site. The distal vent port is positioned at the specimen site. The needle includes a handle that extends radially outward from the elongated body to permit a force to be applied to the needle for directing the needle to the specimen site. The handle includes a vent channel that is in fluid communication with the second lumen. The aspirating needle includes an air reservoir that is open to atmosphere and includes a mechanism for limiting an amount of air that can be delivered to the specimen site by traveling within the second lumen and being discharged through the distal vent port.

An aspirating needle for collecting a specimen includes an elongated body that includes a first lumen and a second lumen. The first lumen is open at both ends for placement at a specimen site to collect and permit aspiration of the specimen from the specimen site. The second lumen terminates at a distal vent port formed along the elongated body such that that when the needle is placed at the specimen site. The distal vent port is positioned at the specimen site. The aspirating needle includes a handle that extends radially outward from the elongated body to permit a force to be applied to the needle for directing the needle to the specimen site. The handle includes a hollow reservoir that is in fluid communication with the second lumen and is constructed to hold a preselected volume of air that is for introduction to the specimen site by traveling within the second lumen and being discharged through the distal vent port. A plunger is provided and has a plunger seal member that is disposed within the reservoir in a sealed manner and moves axially therein to define an inner space between the plunger seal member and the second lumen. The inner space has a variable first volume that defines an amount of air that can be delivered to the specimen site.

In another embodiment, an aspirating needle for collecting a specimen includes an elongated body that includes a first lumen and a second lumen. The first lumen is open at both ends for placement at a specimen site to collect and permit aspiration of the specimen from the specimen site. The second lumen terminates at a distal vent port formed along the elongated body such that that when the needle is placed at the specimen site. The distal vent port is positioned at the specimen site. The aspirating needle includes a handle that extends radially outward from the elongated body to permit a force to be applied to the needle for directing the needle to the specimen site. The handle includes a vent channel that is in fluid communication with the second lumen. A luer lock connector is disposed along the handle and is in fluid communication with the vent channel. The aspirating needle further includes a luer lock syringe that is configured to sealingly attach to the luer lock connector and including a barrel with a slidable plunger for delivering a preselected volume of air that is for introduction to the specimen site by traveling within the second lumen and being discharged through the distal vent port.

Other features and advantages of the present invention will be apparent from the following detailed description when read in conjunction with the accompanying drawings.

### BRIEF DESCRIPTION OF THE DRAWING FIGURES

The foregoing and other features of the present invention will be more readily apparent from the following detailed description and drawings figures of illustrative embodiments of the invention in which:

FIG. 1 is a perspective view of an aspiration needle according to a first embodiment for use at a site that is aspirated to collect a sample, such as a fluid sample of bone marrow;

FIG. 2A is a partial cross-section of the aspiration needle according to the first embodiment showing a plunger member in a first position;

FIG. 2B is a partial cross-section of the aspiration needle according to the first embodiment showing a plunger member in a second position;

FIG. 3 is a perspective view of a portion of a handle of the aspiration needle of FIG. 1 showing a stop feature and the plunger member;

FIG. 4A is a partial cross-section of an aspiration needle according to a second embodiment for use at a site that is aspirated to collect a sample, such as a fluid sample of bone marrow, with a plunger syringe being exploded from a handle of the aspiration needle;

FIG. 4B is a partial cross-section of the aspiration needle of FIG. 4A showing the plunger syringe attached to the handle;

FIG. 5 is a partial cross-section of an aspiration needle according to a third embodiment for use at a site that is aspirated to collect a sample, such as a fluid sample of bone marrow, with a plunger syringe being exploded from a handle of the aspiration needle.

### DETAILED DESCRIPTION OF THE PREFERRED EMBODIMENT

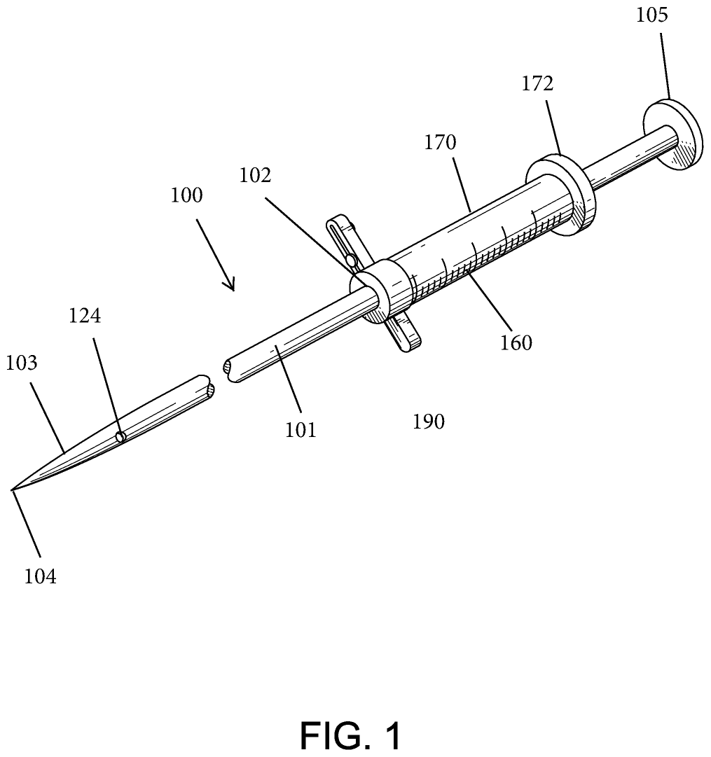

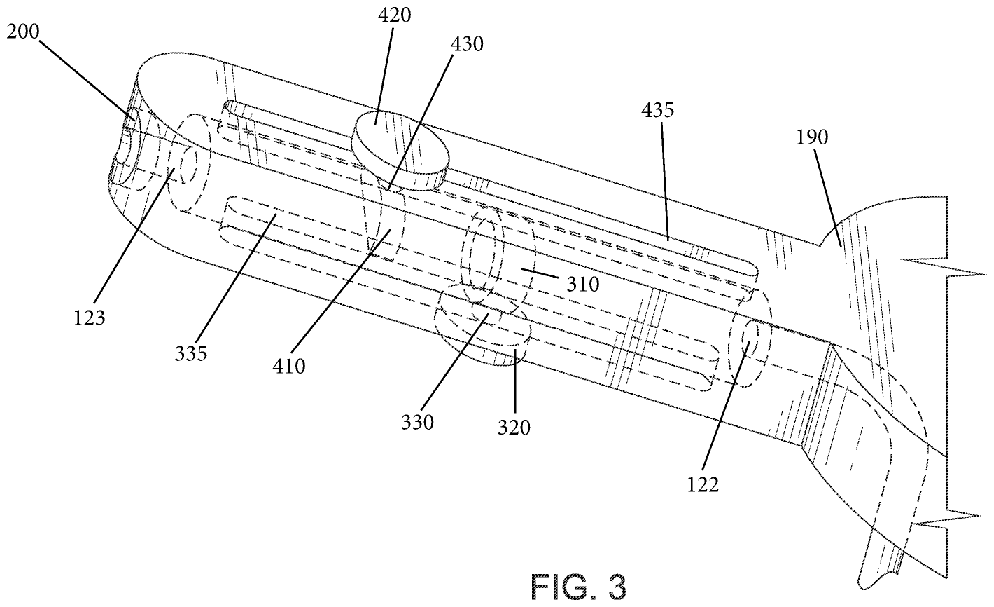

### First Embodiment (FIGS. 1-3)

Referring now to FIGS. 1-3, an aspiration needle 100 according to a first exemplary embodiment is illustrated and is particularly suited for use at a target site, such as one associated with a bone marrow aspiration application. In other words, while the aspiration needle 100 is particularly suited for use in medical applications where aspiration of a local site takes place, it will be understood that the needle 100 is suited for other applications in which equilibration of the aspirated space is desirable. It is contemplated that there are additional non-medical applications for the aspiration needle 100 with one potential application being a manufacturing type application or a laboratory application where equilibration is needed.

As described herein, the needle 100 is configured and intended for coupling to a syringe 160 to form the assembled article shown in FIG. 1.

The aspiration needle 100 is constructed to overcome the deficiencies associated with conventional bone marrow aspiration needles and more specifically, is constructed to minimize the potential negative pressure that develops during the bone marrow collection and aspiration process. In order to achieve this goal, the needle 100 includes a type of “vent” that modulates the pressure in the bone marrow space as the material is withdrawn. In addition, as described herein, the degree (amount) of venting is selectable and is limited due to the presence of an air compartment that holds a prescribed volume of air for venting of the surgical site.

The needle 100 has a body 101 that is defined by a first end 102 that is a proximal end and an opposing second end 104 that is a distal end. The second end 104 (distal end) is the sharp tip end for puncturing the cortical bone during the bone marrow collection procedure. The needle 100 is based on a double lumen design in that the needle 100 includes a first lumen 110 and a second lumen 120 that is proximate the first lumen 110. The first lumen 110 acts as a conduit for material to be withdrawn out of a bone marrow space into the syringe 160, while the second lumen 120 comprises a venting lumen that for allows for venting of the bone marrow space (e.g., in one embodiment, the pressures within the bone marrow space equilibrate with outside pressures). The needle 100 is securely coupled to the syringe 160 using conventional techniques, such as threaded fastening means, generally shown at 171 in the figures. For example, the syringe 160 can be screwed on to the threaded fastening means 171 (i.e., internal threads formed at proximal end of the needle body within a bore). The syringe 160 include a collection chamber 170 (barrel chamber) for collecting the sample that is aspirated through the needle 100. The syringe 160 can have a flange 172 at its proximal end.

The division of the body 101 of the needle 100 can be accomplished in any number of different ways so long as the body 101 is divided into the first lumen 110 and the second lumen 120. For example, the needle 100 can include a dividing or partitioning wall 140 that is formed within the body 101 and serves to partition at least a length of the interior of the body 101 into the first and second lumens 110, 120. It will be appreciated that the wall 140 does not have to evenly divide the interior of the body 101 such that the first and second lumens 110, 120 occupy the same area but rather the first and second lumens 110, 120 can occupy different amounts of areas. Thus, while FIG. 1 shows the wall 140 generally evenly dividing the interior of the body 101 into the first and second lumens 110, 120, this is merely exemplary and illustrative in nature as opposed to being limiting. For example, the second lumen 120 that is associated with performing a venting operation can occupy less area than the first lumen 110 which serves as the conduit for withdrawing the bone marrow or sample material. Another embodiment is one where the divider 140 is eliminated and a tube, possibly flexible, is provided within the body of the needle 101 with one end of the tube exiting at the lumen 120 and the other exiting or connected to the lumen or vent.

The first lumen 110 is thus a generally unobstructed channel that extends from the first end 102 to a location at or close to the second end 104 and therefore, it permits material to be aspirated into the distal second end 104 and withdrawn to the first end 102 in a generally linear manner. Conversely, the second lumen 120 is not constructed to receive material at the bone marrow site (space) but rather, the second lumen 120 is constructed to permit atmospheric air to be delivered to the bone marrow site so as to serve as a vent and allow the pressure within the bone marrow space to equilibrate with outside pressures. e.g., atmospheric pressure.

As will be appreciated, the aspiration event in the first lumen 110 and the removal of the target material at the surgical site generates a negative pressure at the surgical site itself at which the target material is removed. As described herein, venting is made possible due to the positioning of the opening (vent) 124 at the surgical site.

In the illustrated embodiment, the second lumen 120 has a first open end and an opposing open second end, with the first end being proximate or close to the first end 102 of the needle 100 and the second end being proximate or close to the second end 104 of the needle 100. As shown in FIG. 1, the open first end is in the form of a first vent port 122 and the second end is in the form of a second vent port 124. The second vent port 124 is formed in a side surface 103 of the body 101 as opposed to being formed directly at the second end 104. The second vent port 124 is preferably formed in the side surface either at or close to the second end 104 since the second vent port 124 is to be in fluid communication with the bone marrow space when the distal end 104 of the needle 100 is inserted and guided to the bone marrow collection site (space).

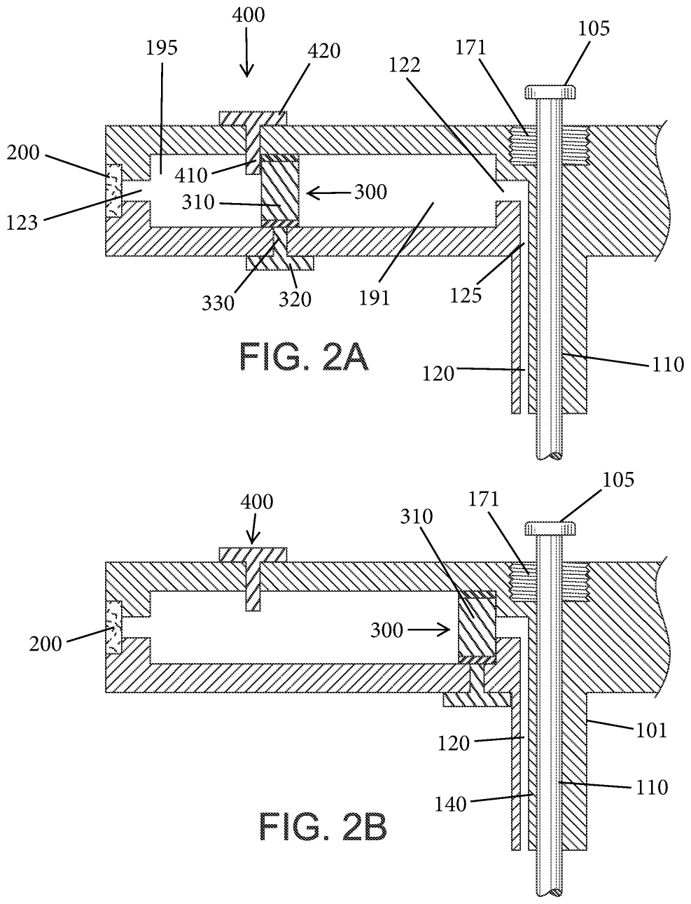

FIGS. 2A and 2B illustrate the needle 100 being used in combination with a stylet 105. The relative areas of the first and second lumens 110, 120 are shown and more particularly, the first lumen 110 occupies significantly more cross-sectional area than the second lumen 120 since the first lumen 110 receives and permits aspiration of the sample to the collection chamber 170 (FIG. 1); while the second lumen 120 performs a venting action and only needs to permit air to pass therealong. The cross-sectional area of the second lumen 120 should be such that it does not produce a resistance to airflow and permits the desired venting action.

It will be appreciated that the ratio of the cross-sectional areas between the first and second lumens 110, 120 is variable depending upon the particular given application; however, the dimensions of the second lumen 120 is such that it does not produce a resistance to airflow but instead vents the air. For example, the cross-sectional area of the second lumen 120 compared to the entire cross-sectional area of the needle 100 can be on the order of between about 1% to about 40% in one embodiment, between 10%-30% in another embodiment; and 15%-25% in another embodiment. However, these values are merely exemplary in nature and are not limiting of the present scope of the present invention in any manner.

The stylet 105 as is known is placed within the needle 100 and has a tip to it which allows the needle 100 to puncture the cortex. The stylet 105 is removed once the needle 100 has penetrated the cortex opening up the first lumen 110 for the aspiration procedure. In this needle design, the stylet 105 will initially sit within the first lumen 110 that carries the material from the bone marrow to the syringe and not in the venting lumen (second lumen 120).

The needle 100 includes a handle 190 that permits and allows the appropriate mechanical usage of the needle 100. During the normal use of the needle 100, the physician or operator has to apply a sufficient force against the needle 100 to drive the needle through tissue and the like in order to locate and place the needle tip 104 at the operative site, e.g., a bone marrow site. More particularly, the stylet 105 is introduced into the first lumen 110 and is advanced therein until it extends beyond the second end 104. As mentioned above, the stylet 105 is used to initially place and locate the needle 100 at the operative site. A significant amount of force is therefore required to direct the needle through tissue and therefore, the handle 190 provides a spot where the operator can hold and apply force against the needle body.

The handle 190 can be located at any number of different locations along the body of the needle 100. For example, the handle 190 can be integrally formed along the length of the lumens 110, 120 or can be attached thereto. The handle 190 likewise can have any number of different shapes and can have an annular shape where it extends around the complete periphery of the needle body or it can be in the form of one or more fingers or tabs that extend outwardly from the needle body (e.g., from lumens 110, 120 or fastening means 130). In FIG. 2, the handle 190 is in the form of a pair of opposing fingers or tabs that are curved or rounded at a distal end to permit the operator to place either two fingers against the two handle tabs or place fingers of two hands against the two handle tabs.

In the illustrated embodiment, the handle 190 is at the proximal end of the needle at a location where the syringe 160 is coupled to the needle 100. As shown, the handle 190 includes the threaded fastening means 171 formed in a cavity or space in which the distal end of the syringe 160 is sealingly and threadingly mated to the needle 100 to form a combined structure.

In the first embodiment, the handle 190 is configured such that it is in fluid communication with the second lumen 120 and more particularly, at least a portion of the vent channel that extends between the first and second vent ports 122, 124 is formed in the handle 190 itself. More specifically, the second lumen 120 includes a vent channel 125 that extends between the first vent port 122, which in this case is formed in the handle 190, and the second vent port 124 which is formed in the side wall of the needle 100.

The handle 190 in the first embodiment includes an air (vent) reservoir 191 which is a hollow space formed in the handle 190. The illustrated air reservoir 191 can extend radially within the handle 190 in that the air reservoir 191 extends from near a perimeter edge of the handle 190 to a location closer to the center needle. The air reservoir 191 is in fluid communication with the second lumen 120 and thus, air within selection regions of the air reservoir 191 can flow to the surgical site under select conditions. The air reservoir 191 can have different cross-sectional shapes, including a cylindrical shape, a square shape, etc.

In FIGS. 1 and 2, the first vent port 122 is formed at an inner end of the air reservoir 190 and at an opposite outer end of the air reservoir 190, there is an outer vent port 123. The outer vent port 123 is directly open to atmosphere. The outer vent port 123 is formed along one surface of the handle 190 and more particularly, can be formed at/along the outer perimeter of the handle 190. The outer vent port 123 can have a filter element 200 for filtering air drawn through the outer vent port 123 and into the air reservoir 191. Any number of traditional air filter materials can be used as the filter element 200.

Unlike the previous design in Applicant's U.S. Pat. No. 7,207,950, which is hereby incorporated by reference in its entirety, the air reservoir 191 is not continuously and freely open to atmosphere, whereby, an unlimited volume of air from the air reservoir 191 is delivered to the surgical site. In contrast, only a controlled, selected volume of air from the air reservoir 191 is deliverable to the surgical site. To achieve this, a plunger mechanism is provided and includes a movable plunger 300 that has a portion that is sealed against an inner wall of the air reservoir 191 and can slidingly travel therein. The movable plunger 300 thus includes a plunger member 310 that seals against the wall of the air reservoir 191.

The plunger member 310 has a complementary shape relative to the shape of the air reservoir 191 to allow the plunger member 310 sealingly seat against the inner wall of the air reservoir 191. For example, when the air reservoir 191 has a cylindrical shape, the plunger member 310 likewise has a cylindrical (barrel) shape. The plunger 300 moves in a linear (axial) manner within the air reservoir 191 (i.e., in a direction toward and away from the center needle body).

The plunger member 310 partitions the air reservoir 191 into two compartments (spaces) of varying volumes depending upon the location of the plunger member 310 within the air reservoir 191. An outer space, generally indicated at 195, is located between the outer vent port 123 at the outer end of the air reservoir 191 and the plunger member 310 and an inner space, generally indicated at 197, is located between the plunger member 310 and the inner end of the air reservoir 190. As mentioned, the volume of each of the outer space 195 and the inner space 197 is variable depending on the location and axial movement of the plunger member 310 within the air reservoir 191. For example, as the plunger member 310 moves in a direction toward the elongated body of the needle, the volume of the inner space 197 decreases and the volume of the outer space 195 increases.

In the illustrated embodiment, the plunger 300 includes an outer slider (actuator) 320 that is connected to the plunger member 310 by means of a connector post or stem 330. The outer slider 320 and post 330 can have a T-shape orientation. The outer slider 320 is accessible by the user along the outer surface of the handle 190. The post 330 passes through and moves within an axial slot 335 that is formed along a bottom surface (underside) of the handle 190 to allow the plunger 300 to move within the air reservoir 191. The outer slider 320 allows the plunger member 310 to be easily moved within the air reservoir 191.

As described herein, the air within the outer space 195 that flows into the outer space through the outer vent port 123 cannot flow to the surgical site due to the sealed plunger member 310 being located between the outer space 195 and the surgical site. In contrast, the air within the inner space 197 can flow unimpeded to the surgical site for venting thereof during the aspiration procedure.

It will be appreciated that the slot 335 is open to atmosphere; however, the inner space 197 still defines the maximum volume of air that can be delivered to the surgical site. As will be appreciated, the air within the inner space 197 and the outside (atmosphere) will be at equilibrium prior to performing the procedure. For example, if the plunger member 310 is located within the air reservoir 191 so that the inner space 197 holds two units of air, then the controlled amount of air that can be delivered to the surgical site for venting is this two units of air. This results since when negative pressure is created at the surgical site due to aspiration of the material from the surgical site, the negative pressure at the surgical site results in air from the second lumen 120 and the air reservoir 191 being drawn downward to the surgical site (to the negative pressure). This also results in the plunger member 310 being drawn inward toward the needle body in the center of the handle. This movement is shown in FIGS. 2A and 2B and in particular, the plunger member 310 moves to the right in FIG. 2A and closes off the first vent port 122 as shown in FIG. 2B when it moves to a maximum closed position. Once the plunger member 310 is in the position shown in FIG. 2B, all of the air within the air reservoir 191 is evacuated and delivered to the surgical site. It will also be appreciated that in the FIG. 2B position of the plunger member 310, the slot 335 is closed off and no atmospheric air can flow through the slot 335. This action results in a user defined, discrete volume of air being delivered to the surgical site as opposed to an unlimited amount of air.

As described herein, in some application, the plunger member 310 may not move completely to position in FIG. 2B but instead may only partially move thereto. In such situations, it is still true that only a discrete, limited amount of air can be delivered to the surgical site due to equilibrium being achieved between the inner space 197 and the outside. In the event that strong negative pressure is realized at the surgical site, more venting air will be pulled from the second lumen 120 and air reservoir 191 to the surgical site and this, will result in the plunger member 310 moving to the position shown in FIG. 2B. Thus, the initial volume of air in the inner space 197 defines the volume of vented air that is available to deliver to the specimen site.

The handle 190 also can include a plunger stop feature in the form of a movable (sliding) stop 400. In the illustrated embodiment, the plunger stop 400 includes an outer slider (actuator) 420 that is connected to a stop member 410 by means of a connector post 430. The outer slider 420 and post 430 can have a T-shape orientation. The outer slider 420 is accessible by the user along the outer surface of the handle 190. The post 430 passes through and moves (slides) within an axial slot 435 that is formed along a bottom surface (underside) of the handle 190 to allow the plunger 400 to move within the air reservoir 191. The outer slider 420 allows the plunger member 410 to be easily moved within the air reservoir 191.

The plunger stop 400 is designed to provide a selectable, prescribed position for the plunger 300 within the air reservoir 191. Each of the top and bottom surfaces of the handle 190 can include volumetric graduations (indicia) that allows the user to easily and quickly position the plunger 300 at a desired location, thereby defining a maximum volume of air that can be delivered to the surgical site. The plunger stop 400 is thus placed at a desired position/location within the air reservoir 191 and then the plunger 300 itself is moved to the left (in FIGS. 2A and 2B) until it contacts the plunger stop 400. Once it contacts the plunger stop 400, the plunger 300 is properly positioned within the air reservoir 191.

It will also be appreciated that the plunger stop 400 can have a lock feature to allow it to be locked in place. Any number of traditional locking mechanisms can be used.

The volumetric graduations along the handle allows for the user to easily position the plunger 300 within the air reservoir 191 so that a defined volume of air is provided within the inner space 197 of the air reservoir 191. As mentioned, this defined volume of air represents the available air for venting the surgical site during the aspiration procedure. Most scales (volumetric graduations) on the barrel of the syringe are traditionally in mL (milliliters) or cc (cubic centimeters). It will be appreciated that there can be a correlation between the amount of material aspirated by the needle 100 and the volume of air that is delivered to the surgical site as part of the venting. In some applications, there can be a 1:1 relationship between the amount of material aspirated and the amount of air delivered to the surgical site. For example, an aspiration of 2 cc of material (e.g., bone marrow from the surgical site can result in 2 cc of air being delivered to the surgical site as part of the venting procedure. A correlation table setting forth the correlation between the amount of material being aspirated and the amount of air delivered to the surgical site can be created to provide guidance to the use. For example, if the user desires to aspirate 3 cc of bone marrow, the correlation table can provide the user with a corresponding amount of air that should be made available for venting (by positioning the plunger 300 at a set location within the air reservoir 191).

Alternatively, in a different embodiment, the location of the outer vent port 123 and filter 200 can be changed and instead, these elements can be located along the top surface (top wall) or bottom surface (bottom wall) of the handle 190. In this alternative embodiment, the plunger for use in the air reservoir 191 can have a traditional shape in that it has a plunger member 310 at one end and a shaft that terminates at a top member at the other end. The top member is the part of the plunger that is accessible to the user and is pushed/pulled by the user to change the position of the plunger. In this design, the location of the outer vent port 123 and the filter elements instead is an open hole through which the shaft of the plunger passes and the top member is located outside the handle 190. The plunger is moved axially within the air reservoir 191.

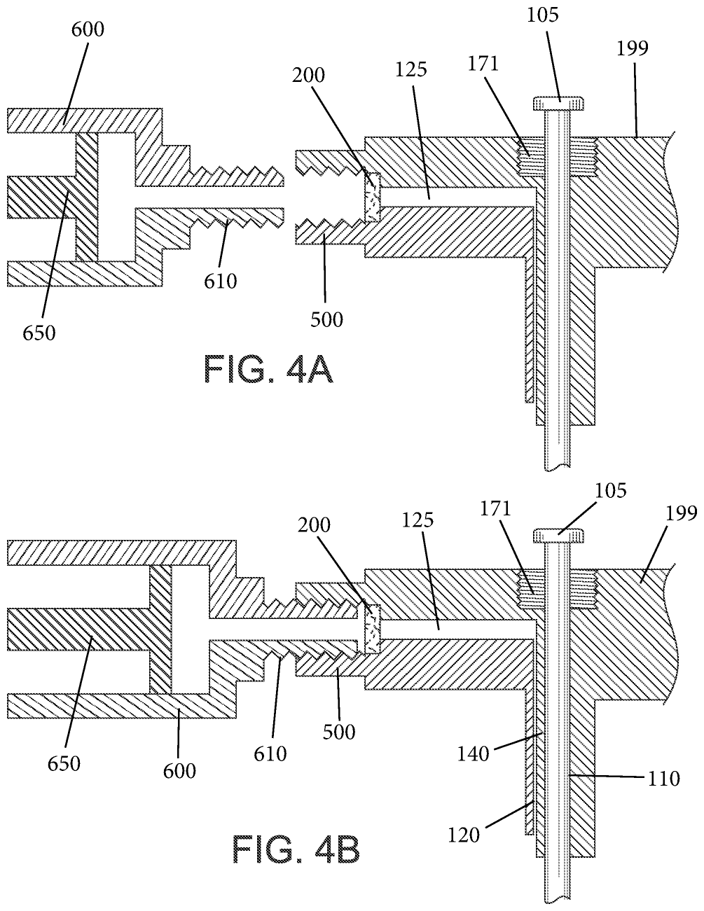

### Second Embodiment (FIGS. 4A and 4B)

The second embodiment has a handle 199 that is similar but modified relative to the handle 190.

In this embodiment, a vent channel 125 is formed in the handle 199 and is in fluid communication with the second lumen 120. At one end (outer end) of the vent channel 125, the filter element 200.

An outer end of the handle 199 also includes a luer lock connector 500. The luer lock connector 500 is configured to mate with a luer lock syringe 600 in conventional manner (by screwing the syringe 600 onto the luer lock connector 500). In particular, the luer lock syringe 600 has a luer connector tip 610 that screws onto the luer lock connector 500 as shown in FIG. 4B.

A traditional syringe plunger 650 is provided within the luer lock syringe 600.

The luer lock syringe 600 is a traditional syringe and includes volumetric graduations (scales) to allow for the user to easily pull in a certain volume of air into the barrel of the syringe 600 by retracting the syringe plunger 650 within the barrel. Like the first embodiment, the air pulled into the barrel of the syringe 600 represents the available air for venting the surgical site during the aspiration procedure. It will be appreciated that the user draws air into the syringe barrel and then connects the syringe 600 to the luer lock connector 500. Once connected, the available venting air is the air that is contained in the syringe barrel and the vent channel 125 and second lumen 120.

As in the first embodiment, as aspiration occurs, air is delivered to the surgical (specimen) site for venting thereof and this can result in movement of the syringe plunger 650 within the barrel depending upon the amount of air needed for venting.

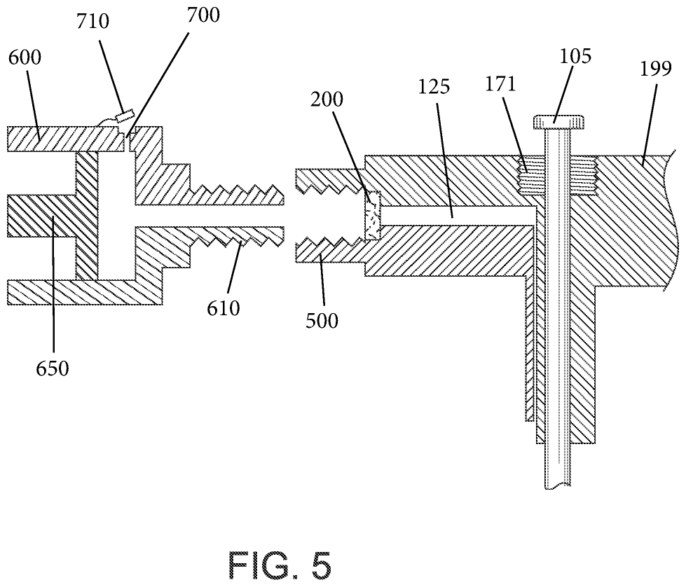

### Third Embodiment (FIG. 5)

As shown in FIG. 5, it is also possible for the luer lock syringe 600 to have an openable and closeable vent port 700 along the barrel near the distal tip to allow for controlled venting of the barrel to atmosphere. For example, in the event that the plunger 650 within the barrel moves all the way to the right due to delivery of the available volume of air, the barrel vent port 700 can be opened and the plunger can be retracted to a desired location, thereby pulling in atmospheric air into the open space created in front of the plunger 650. In this way, additional air can be controllably added to the barrel for an added source of air to deliver to the specimen site. The vent port 700 in the side of barrel at or proximate its distal tip can be a simple hole with a movable port cover or cap or plug 710 that seals the vent port 700 when no venting of the barrel is desired. The cap 710 can be hinged or otherwise attached to the barrel. In this location of the barrel vent port 700, when the plunger is retracted, air is pulled into the barrel when the barrel vent port 700 is open (when the syringe 600 remains connected to the handle).

In both the first and second embodiments, the amount of air that can be delivered to the surgical site for venting is limited unlike in the previous patent which had continuous atmospheric venting. The present embodiments thus limit the volume of air entering the marrow space (surgical site) while still providing air to displace the marrow that is aspirated.

It will also be appreciated that there are other mechanism that serve to limit the amount of air that can be delivered to the specimen site within the spirit of the present disclosure. In other words, the present disclosure provides systems and mechanism by which the amount of venting air that can be delivered is controlled (generally closed system) as opposed to having an open system in which air can be freely pulled from atmosphere and delivered to the specimen site.

While exemplary drawings and specific embodiments of the present invention have been described and illustrated, it is to be understood that the scope of the present invention is not to be limited to the particular embodiments discussed. Thus, the embodiments shall be regarded as illustrative rather than restrictive, and it should be understood that variations may be made in those embodiments by workers skilled in the art without departing from the scope of the present invention as set forth in the claims that follow, and equivalents thereof. In addition, the features of the different claims set forth below may be combined in various ways in further accordance with the present invention.

What is claimed is:

1. An aspirating needle for collecting a specimen comprising:
an elongated body that includes a first lumen and a second lumen, the first lumen being open at both ends for placement at a specimen site to collect and permit aspiration of the specimen from the specimen site, the second lumen terminating at a distal vent port formed along the elongated body such that that when the needle is placed at the specimen site, the distal vent port is positioned at the specimen site:
a handle that extends radially outward from the elongated body to permit a force to be applied to the needle for directing the needle to the specimen site, wherein the handle includes a hollow reservoir that is in fluid communication with the second lumen and is constructed to hold a preselected volume of air that is for introduction to the specimen site by traveling within the second lumen and being discharged through the distal vent port; and
a plunger having a plunger seal member that is disposed within the reservoir in a sealed manner and moves axially therein to define an inner space between the plunger seal member and the second lumen, the inner space having a variable first volume that defines an amount of air that can be delivered to the specimen site.

2. The aspirating needle of claim 1, wherein the handle includes a top surface and a bottom surface, the bottom surface including a bottom axial slot through which the plunger passes, the plunger moving axially within the bottom axial slot to vary the first volume of the inner space.

3. The aspirating needle of claim 2, wherein the plunger includes a slider located outside of the handle and a stem that passes through the bottom axial slot and attaches the slider to the plunger seal member.

4. The aspirating needle of claim 2, wherein the bottom axial slot is open to atmosphere.

5. The aspirating needle of claim 1, further including an outer vent port that is formed along an outer end of the handle at or proximate an outer end of the reservoir, the outer vent port being open to atmosphere and including a filter element.

6. The aspirating needle of claim 5, wherein an outer space is formed between the plunger seal member and the outer vent port.

7. The aspirating needle of claim 1, further including a plunger stop, the plunger stop being disposed within the reservoir and moving axially therein.

8. The aspirating needle of claim 1, wherein the handle includes a top surface and a bottom surface, the top surface including a top axial slot through which the plunger stop passes, the plunger stop moving axially within the top axial slot to vary a location of the plunger stop.

9. The aspirating needle of claim 8, wherein the plunger stop includes a slider located outside of the handle and a stem that passes through the top axial slot and attaches the slider to the plunger stop.

10. The aspirating needle of claim 7, wherein the plunger stop is disposed between the plunger and an outer end of the hollow reservoir.

11. The aspirating needle of claim 7, wherein the plunger stop includes a lock mechanism for temporarily locking the plunger stop in a preselected position within the reservoir.

12. The aspirating needle of claim 1, wherein the handle includes volumetric graduations that correspond to dosage volumes within the reservoir.

13. The aspirating needle of claim 1, wherein the needle includes an atmospheric vent that includes a filter in fluid communication with the atmospheric vent.

14. The aspirating needle of claim 1, wherein the handle is perpendicular to the elongated body.

15. The aspirating needle of claim 1, wherein the plunger seal member has a cylindrical shape and the reservoir has a cylindrical shape, wherein the plunger includes a slider located outside of the handle and connected to the plunger seal member by a stem that protrudes outward from a side of the plunger seal member.

16. An aspirating needle for collecting a specimen comprising:
an elongated body that includes a first lumen and a second lumen, the first lumen being open at both ends for placement at a specimen site to collect and permit aspiration of the specimen from the specimen site, the second lumen terminating at a distal vent port formed along the elongated body such that that when the needle is placed at the specimen site, the distal vent port is positioned at the specimen site;
a handle that extends radially outward from the elongated body to permit a force to be applied to the needle for directing the needle to the specimen site, wherein the handle includes a vent channel that is in fluid communication with the second lumen; and
a luer lock connector that is disposed along the handle and is in fluid communication with the vent channel.

17. The aspirating needle of claim 16, further including a luer lock syringe that is configured to sealingly attach to the luer lock connector and including a barrel with a slidable plunger for delivering a preselected volume of air that is for introduction to the specimen site by traveling within the second lumen and being discharged through the distal vent port.

18. The aspirating needle of claim 16, wherein at least a section of the vent channel is formed perpendicular to the second lumen.

19. The aspirating needle of claim 16, wherein the luer lock connector is formed perpendicular to the second lumen.

20. The aspirating needle of claim 16, wherein the first lumen and the second lumen run parallel to one another within the elongated body.

21. The aspirating needle of claim 16, wherein the vent channel has a first end at an interface with the luer lock connector and an opposite second end that intersects the second lumen.

22. The aspirating needle of claim 21, wherein the first end of the vent channel has a filter.

23. An aspirating needle for collecting a specimen comprising:
an elongated body that includes a first lumen and a second lumen, the first lumen being open at both ends for placement at a specimen site to collect and permit aspiration of the specimen from the specimen site, the second lumen terminating at a distal vent port formed along the elongated body such that that when the needle is placed at the specimen site, the distal vent port is positioned at the specimen site;
a handle that extends radially outward from the elongated body to permit a force to be applied to the needle for directing the needle to the specimen site, wherein the handle includes a vent channel that is in fluid communication with the second lumen; and
an air reservoir that is at least selectively open to atmosphere and includes a mechanism for limiting an amount of air that can be delivered to the specimen site by traveling within the second lumen and being discharged through the distal vent port for venting of the specimen site.
